(19) United States
(12) Patent Application Publication
(10) Pub. No.: US 2023/0395197 A1
(43) Pub. Date: Dec. 7, 2023

(54) METHOD AND SYSTEM FOR EARLY EFFICIENT DETECTION OF CO-EVOLUTIONARY SITES IN EVOLVING BIO-NETWORKS

(71) Applicant: UNIVERSITY OF VIRGINIA PATENT FOUNDATION, Charlottesville, VA (US)

(72) Inventors: Christopher L. Barrett, Charlottesville, VA (US); Christian M. Reidys, Blacksburg, VA (US)

(73) Assignee: UNIVERSITY OF VIRGINIA PATENT FOUNDATION, Charlottesville, VA (US)

(21) Appl. No.: 18/246,372

(22) PCT Filed: Sep. 30, 2021
(86) PCT No.: PCT/US2021/052999

§ 371 Date: Mar. 23, 2023

Related U.S. Application Data
(60) Provisional application No. 63/085,949, filed on Sep. 30, 2020.

(51) Int. Cl. G16B 40/30; G16B 30/10

(52) U.S. Cl. CPC G16B 40/30; G16B 30/10; G16B 45/00

(57) ABSTRACT

A method and system are disclosed for efficient early detection of co-evolutionary sites among genomic sequences. Exemplary embodiments extract/approximate a data motif complex of a given data set wherein the extraction procedure can be performed using at least two steps. One step is construction of a vertex set of the data motif complex by identifying data sites with high informational variation. Another step is construction of higher dimensional simplices which systematically represent informational patterns within the data set. The method and system can be implemented as a computer-implemented software pipeline as described herein. An exemplary application can rapidly recognize key or critical mutational blocks in viral SARS-CoV-2 genomic data.

### RELATED APPLICATION

This application is a U.S. national stage application under 35 U.S.C. § 371 for International Patent Application No. PCT/US2021/052999, filed on Jul. 28, 2020, which claims the benefit under 35 U.S.C. § 119 of U.S. Provisional Patent No. 63/085,949 filed on Sep. 30, 2020, the entire contents of each are hereby incorporated by reference in their entirety.

### FIELD

A method and system are disclosed for efficient early detection of co-evolutionary sites among aligned genomic sequences. For example, variants of clusters of co-evolutionary sites can be detected within a collection of coded sequences based on inter-site dependencies. Exemplary applications include, for example, determining indications and warnings of possible pandemic warning indications for assisting with predicting and planning a response to global phenomena such as biocomplex pandemics, and/or assessing genetic make-up to assess where on a genome a putative fitness exists when evaluating fitness of selected seed types for specified conditions such as draught resistance.

### BACKGROUND INFORMATION

Humanity is faced with dynamically managing a host of critical global challenges that simultaneously catalyze, manifest, and interact on many levels—from molecular to societal and beyond. Ecological and economic crises can illustrate such phenomena, but the on-going novel COVID-19 pandemic provides a clear, current, and urgent example of extreme-scale biocomplexity. This crisis demands conceptual, mathematical innovation that improves our ability to comprehend and address it productively on multiple levels. A key question involves representing these relevant and diverse levels and measuring how they interact, such as with the case of the COVID-19 pandemic as an exemplar. New algebraic homology and information theories are needed to be coupled with rigorous algorithmic and simulation science theories that will lead to scalable simulations of multi-layer contagion phenomena. Novel mathematical constructs that quantify feedback loops among multiscale interactions need to be developed. The resulting technologies will lead to improved pandemic planning and response.

New mathematical and computational theories and engineering principles need to be developed with the goal of uncovering fundamental features of multiscale interacting and evolving bio-networks. COVID-19 is a paradigmatic example for which there is a need to discover how social policies affect the mutant composition of viral populations and their evolutionary capacity, such as a population's closeness to “bad” mutants to assist in mitigating or avoiding the global burden of infectious diseases. As noted by Fineberg and Wilson (Science 2009), few situations illustrate the salience of the chain “theory to science to policy” more dramatically than a global epidemic.

Additional information is set forth in the following documents, all of which are hereby incorporated by reference in their entireties:
[1] S T Ali et al. “Serial interval of SARS-CoV-2 was shortened over time by nonpharmaceutical interventions”. In: Science 369.6507 (2020), pp, 1106-1109.
[2] C Barrett et al. “EpiSimdemics: an efficient algorithm for simulating the spread of infectious disease over large realistic social networks”. In: SC'OS: Proceedings of the 2008 ACM/IEEE Conf on Supercomputing. IEEE. (2008), pp. 1-12.
[3] C Barrett et al. “Generation and analysis of large synthetic social contact networks”. In: Winter Simulation Conference. Winter Simulation Conference. (2009), pp. 1003-1014.
[4] C Barrett et al. “Multiscale feedback loops in SARS-CoV-2 viral evolution”. In: J. Comput. Biol (2020).
[5] K R Bisset et al. “EpiFast: a fast algorithm for large scale realistic epidemic simulations on distributed memory systems”. In: Proceedings of the 23rd International Conference on Supercomputing. (2009), pp. 430-439.
[6] K R Bisset et al. “Indemics: An interactive high-performance computing framework for dala-intensive epidemic modeling”. In: ACM Transactions on Modeling and Computer Simulation (TOMACS) 24.1 (2014), pp. 1-32.
[7] A C Bura, Q He, and C M Reidys. “Weighted Homology of Bi-Structures over Certain Discrete Valuation Rings, Mathematics 2021, 9(7), 744; https://doi.org/10.3390/math9070744, received: 23 Feb. 2021/Revised: 27 Mar. 2021/Accepted: 29 Mar. 2021/Published: 31 Mar. 2021
[8] R Eletreby et al. “The effects of evolutionary adaptations on spreading processes in complex networks”. In: Proceedings of the National Academy of Sciences 117 (2020), p. 201918529.
[9] S Eubank et al, “Modelling disease outbreaks in realistic urban social networks”. In: M/we 429.6988 (2004), pp, 180-184.
[10] T J X Li and C M Reidys. “On an enhancement of RNA probing data using information theory”. In; Algorithms for Molecular Biology 15.1 (2020), pp. 1-22.
[11] D Machi et al. Scalable Epidemiological Workflows to Support COVID-19 Planning and Response. Tech. rep. Biocomplexity Institute, University of Virginia. (2020).
[12] M Marathe and A Vullikanti. “Computational Epidemiology”. In: Communications of the ACM 56.7 (2013), pp. 88-96.
[13] M S Waterman. “Secondary structure of single-stranded nucleic acids”. In: Adv. Math. Suppl. Studies I (1978), pp. 167-212.
[14] Brown, C., Vostok, J., Johnson, H., et al., 2021. Outbreak of SARS-CoV-2 infections, including covid-19 vaccine breakthrough infections, associated with large public gatherings. Morbidity and Mortality Weekly Report 70 (31), 1059-1062, DOI: http://dx.doi.org/10.15585/mmwr.mm7031 e2.
[15] Elbe, S., Buckland-Merrett, G., 2017. Data, disease and diplomacy: Gisaid's innovative contribution to global health. Global Challenges 1 (1), 33-46.
[16] Hodcroft, E. B., Zuber, M., Nadeau, S., Vaughan, T. G., Crawford, K. H. D., Althaus, C. L., Reichmuth, M. L., Bowen, J. E., Walls, A. C., Corti, D., Bloom, J. D., Veesler, D., Mateo, D., Hernando, A., Comas, I., Gonzalez Candelas, F., Stadler, T., Neher, R. A., 2021. Spread of a SARS-CoV-2 variant through Europe in the summer of 2020. Nature 595 (7869), 707-712.
[17] Katoh, K., Misawa, K., Kuma, Miyata, T., 2002. Mafft: a novel method for rapid multiple sequence alignment based on fast fourier transform. Nucleic acids research 30 (14), 3059-3066.
[18] Shu, Y., McCauley, J., 2017. Gisaid: Global initiative on sharing all influenza data—from vision to reality. Eurosurveillance 22 (13). WHO, 2021. WHO announces simple, easy-to-say labels for SARS-CoV-2 variants of interest and concern. www.who.int.
[19] Sievers, Fabian, and Desmond G. Higgins. “Clustal omega.” Current protocols in bioninformatics 48, no. 1 (2014): 3-13.
[20] Johnson, Mark Irena Zaretskaya, Yan Raytselis, Yuri Merezhnk, Scott McGinnis, and Thomas L. Madden. “NCB′ BLAST: a better web interface.” Nucleic acids research 36, no. suppl_2 (2008): W5-W9.
[21] Finn, Robert D., Jody Clements, and Sean R. Eddy. “HMMER web server: interactive sequence similarity searching,” Nucleic acids research 39, no. suppl 2 (2011): W29-W37.

All of the foregoing documents, and all documents listed throughout the following discussion, are hereby incorporated by reference in their entireties.

### SUMMARY

A method is disclosed for efficient early detection (e.g., for indications and warnings) of co-evolutionary sites among aligned genomic sequences, the method comprising: filtering columns of aligned coded sequences wherein evolutionary activity satisfies an evolutionary diversity threshold (d); determining a pair-wise column P-distance matrix for remaining columns of the matrix subject to the evolutionary diversity threshold (d); performing clustering on the remaining columns using the P-distance matrix; and extracting an m-ary approximation of a co-evolution data motif complex structure, the extracting or approximating including: constructing a vertex set of the data motif complex by identifying data sites with specified high informational variation; constructing specified high dimensional simplices which systematically represent key or critical, informational patterns within the data set; and determining and outputting collections of sites within the coded sequences that are acted upon as blocks by selection pressure based on the key or critical, informational patterns.

A system is also disclosed for efficient early detection (e.g., for indications and warnings) of co-evolutionary sites among aligned genomic sequences, the system comprising a computer programmed to perform the steps of: filtering columns of aligned coded sequences wherein evolutionary activity satisfies an evolutionary diversity threshold (d); determining a pair-wise column P-distance matrix for remaining columns of the matrix not subject to the evolutionary diversity threshold (d); performing clustering on the remaining columns using the P-distance matrix; and extracting an m-ary approximation of a co-evolution data motif complex structure, the extracting or approximating including: constructing a vertex set of the data motif complex by identifying data sites with specified high informational variation; contracting specified high dimensional simplices which systematically represent informational patterns within the data set; and determining collections of sites within the coded sequences that are acted upon as blocks by selection pressure based on the key or critical, informational patterns; and a display for outputting a detected variant.

### BRIEF DESCRIPTION OF THE DRAWINGS

Exemplary embodiments of the present invention will be described with respect to the figures, wherein like elements are designated by like numerals, and wherein:

FIG. 1A is an exemplary research workflow diagram applied in accordance with exemplary embodiments of the present invention;

FIG. 1B is an exemplary implementation of a process of the present invention, involving a data motif complex construction and flow diagram in a specific exemplary application to a collection of SARS-CoV-2 genomes over a given period of time at a specified geographical location;

FIG. 1C is an exemplary hardware/software pipeline implementation of the FIG. 1B process for a generalized application;

FIG. 2A is an exemplary illustration of a p-calculation;

FIGS. 2B-1 and 2B-2 are an example heat map of p-values for P(i,j) of active sites, July 2021, USA;

FIG. 3A shows a known Alpha variant mutational clusters (November 2020, England), wherein mutations on a same height belong to the same cluster;

FIG. 3B is cumulative infections and deaths (total and by strain) for New York (I,II) and California (IIIJV), respectively, under the lock-down scenario (solid lines) or a base case with no mitigation (dashed lines);

FIG. 4 shows newly emerging active mutations and their relative cluster distributions for various variants in England, November 2020 to June 2021, wherein a total number of clusters for each month is held constant (5) by introducing empty clusters if in a particular month the number of clusters generated is smaller than 5; each colored portion of a vertical bar represents the ratio of actively emerging mutations corresponding to various variants within each given cluster, and if a mutation is contained in multiple variants then it is counted with multiplicity; the right side lists the genomic positions appearing in the highlighted regions corresponding to the Alpha variant;

FIG. 5 shows newly emerging active mutations and their relative cluster distributions for various variants in the USA, February 2021 to July 2021, wherein a t total number of clusters is again kept constant; each colored portion of a vertical bar represents the ratio of actively emerging mutations corresponding to various variants within each given cluster, and if a mutation is contained in multiple variants then it is counted with multiplicity; the right and left sides list the genomic positions appearing in the highlighted regions corresponding to the Delta variant and the genomic sub-lineage AY.3;

FIG. 6 shows Mu variant mutational clusters (July 2021, South America), wherein mutations on the same height belong to the same cluster; and

FIG. 7 shows newly emerging active mutations and their relative cluster distributions for various variants in South America, February 2021 to July 2021, wherein a total number of clusters is again kept constant; each colored portion of a vertical bar represents a ratio of actively emerging mutations corresponding to various variants within each given cluster, and if a mutation is contained in multiple variants then it is counted with multiplicity; the right and left list the genomic positions appearing in the highlighted regions corresponding to the Lambda and Mu variants.

### DETAILED DESCRIPTION

A motif complex in accordance with exemplary embodiments described herein is used to detect what amounts to be maximal collections of aligned code sequenced sites that experience a phenomenon referred to herein as selection pressure. In exemplary genomic embodiments, this selection pressure manifests by a small number of mutational constellations that appear as distinguished patterns within an applied multiple sequence analysis (MSA). The exemplary method cannot rule out that only a core of sites is directly relevant for an underlying functionality, while its complement is “carried along” by founder effect or other mechanisms. However, it is virtually impossible for a motif to emerge at random, as discussed herein, and an identification of cores is manageable because motifs consist typically of a limited number of sites. Despite this, not all motifs result in variants of high consequence (VOCs) or variants of interest (VOIs), as this depends on viral dynamics and external factors, such as selection pressures exerted via vaccinations or social distancing.

The motif complex can, by construction, not draw any conclusions as for which of these motifs will constitute a problem later. This can be achieved by detailed biological analysis. Short of biological analysis, the identification of these motifs provides critical and timely value by a process.

Even when mapped onto specific VOCs an VOIs, the motifs can provide insight in how characteristic mutations organize, which in itself aids the biological analysis

Thus, according to exemplary embodiments a method is disclosed for efficient early detection (e.g., for indications and warnings) of co-evolutionary sites among aligned genomic sequences, the method including filtering columns of aligned coded sequences wherein evolutionary activity satisfies an evolutionary diversity threshold (d); determining a pair-wise column P-distance matrix for remaining columns of the matrix subject to the evolutionary diversity threshold (d); performing clustering (e.g., k-means and/or HCS-clustering) on the remaining columns using the P-distance matrix; and extracting an m-ary approximation of a co-evolution data motif complex structure, the extracting or approximating including: constructing a vertex set of the data motif complex by identifying data sites with specified high informational variation; constructing specified high dimensional simplices which systematically represent key or critical, informational patterns within the data set; and determining and outputting collections of sites within the coded sequences that are acted upon as blocks by selection pressure based on the key or critical, informational patterns.

The method for early detection can include aligning a plurality of coded sequences using a multiple sequence analysis (MSA).

The method for early detection can include determining pair-wise column P- and/or J-distance matrices for remaining columns of the matrix subject to the evolutionary diversity threshold (d).

The method for early detection can include detecting variants for indication and warnings during biologic analysis.

The method for early detection can include assessing a collection represented by a population of RNA nucleotide sequences associated with positive samples of an infectious population.

The method for early detection can include assessing a collection represented by a population of DNA sequences associated with positive samples of an infectious population.

The method for early detection can include implementing the method as a software pipeline.

The method for early detection can include recognizing maximal critical blocks within variants in viral SARS-CoV-2 genomic data.

The method for early detection can include each cluster being interpreted as a disjoint maximal simplex.

A system for efficient early detection (e.g., for indications and warnings) of co-evolutionary sites among aligned genomic sequences can include a computer programmed to perform the steps of: filtering columns of aligned coded sequences wherein evolutionary activity satisfies an evolutionary diversity threshold (d); determining a pair-wise column P-distance matrix for remaining columns of the matrix not subject to the evolutionary diversity threshold (d); performing clustering (e.g., k-means and/or HCS-clustering) on the remaining columns using the P-distance matrix; and extracting an m-ary approximation of a co-evolution data motif complex structure, the extracting or approximating including: constructing a vertex set of the data motif complex by identifying data sites with specified high informational variation; contracting specified high dimensional simplices which systematically represent informational patterns within the data set; and determining and outputting (e.g., via a general-user interface (GUI) and/or display) collections of sites within the coded sequences that are acted upon as blocks by selection pressure based on the key or critical (e.g., pursuant identified or specified threshold for a given application that can, for example, be learned via a machine learning and/or an interactive approach), informational patterns; and a display (e.g., GUI) for outputting a detected variant.

The system for early detection of variants can include the computer being programmed to perform the step of: aligning a plurality of coded sequences using a measurement systems analysis (MSA).

The system for early detection can include the computer being programmed to perform the step of: detecting variants for indication and warnings facilitating effective biologic analysis.

The system for early detection can include the computer being programmed to perform a step of: assessing a collection represented by a population of RNA nucleotide sequences associated with positive samples of an infectious population.

The system for early detection can include the computer being programmed to perform a step of: assessing a collection represented by a population of DNA sequences associated with positive samples of an infectious population.

The system for early detection can include the computer being programmed to perform a step of: implementing the method as a software pipeline.

The system for early detection can include the computer being programmed to perform a step of: detecting critical mutational blocks in viral SARS-CoV-2 genomic data.

Study of infectious disease outbreaks, such as the current COVID-19 pandemic, forces confrontation with among other things, the evolutionary properties of the pathogen; the immunological properties of the host; apprehension, reasoning, and behavior of individuals; the socially-driven interaction networks of individuals and infrastructures; the collection, interpretation, and dissemination of information about the epidemic; and governmental decision-making, policy, law and enforcement. Globalization, climate change, and ecological pressures are likely to enhance the risk of another pandemic. A mathematical path to consilience is exploited herein to create a flexible and evocative, yet rigorous, understanding of interaction systems among such levels. These systems span multiple social, biological, economic and political networks that vary in scale: on the one hand, they include global networks of macroscopic agents such as individual humans, companies, countries, etc. while, on the other hand, they include molecular networks such as viral genomic populations amongst susceptible hosts, or fragments of a viral genome within a single infected host cell.

On the level of macroscopic scales, the classical approach to, for instance, contagion dynamics, encompasses stochastic processes or PDE models that, while useful, do not capture adequately the complex behavior of these systems. For microscopic interaction scales, at the level of molecules, current theories are centered around coding sequences and their effects on protein structure. The impact of the genotype/phenotype change within the evolutionary landscape is not an integral part of the analysis. A plethora of interactions and couplings across these different scales further complicates the problem. This state of affairs calls for new mathematics that captures the behavior of such cross-scale, massively-interacting evolving bio-networks.

It has been discovered that algorithmic and structural mathematical theories and simulations rooted in homological algebra and information theory when combined with data-driven causal models can provide a deeper understanding of multilayered evolving bio-networks for significant problem/solution applications. Such approaches eschew analysis of every complexity of the local structures for particular embodied interactions without losing descriptive richness and indeed providing explanatory insight- and instead focus on the qualities of such couplings.

COVID-19 provides an unprecedented opportunity to study multi-layer evolving networks. The pandemic is likely to leave a fundamental impression on our society for decades to come. Data, policies and methods being developed by individuals and institutions can provide the much needed basis for our work. Furthermore, methods and systems resulting from the present disclosure can lead to tangible and quantitative impact on the social, economic and health burden that COVID-19 imposes. A powerful new paradigm based on an algebraic topology framework that captures the notion of continuity in molecular evolution can be combined with information theoretic methods such as transfer entropies to provide a meaningful concept for quantifying information flow among the different network scales.

Data- and theory-driven computer simulations in conjunction with statistical and machine-learning techniques enable controlled epidemiological experiments that tire otherwise impossible to carry out for ethical or practical reasons.

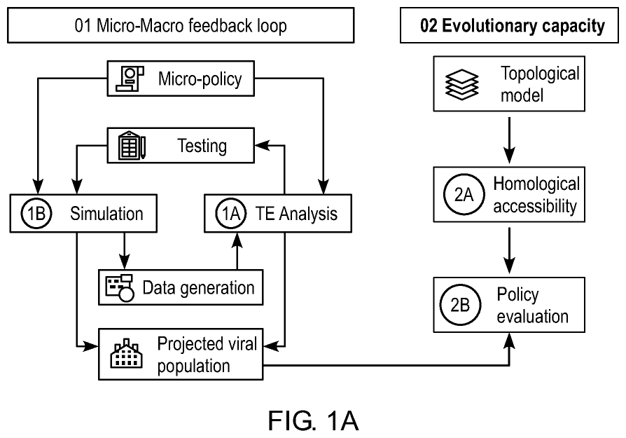

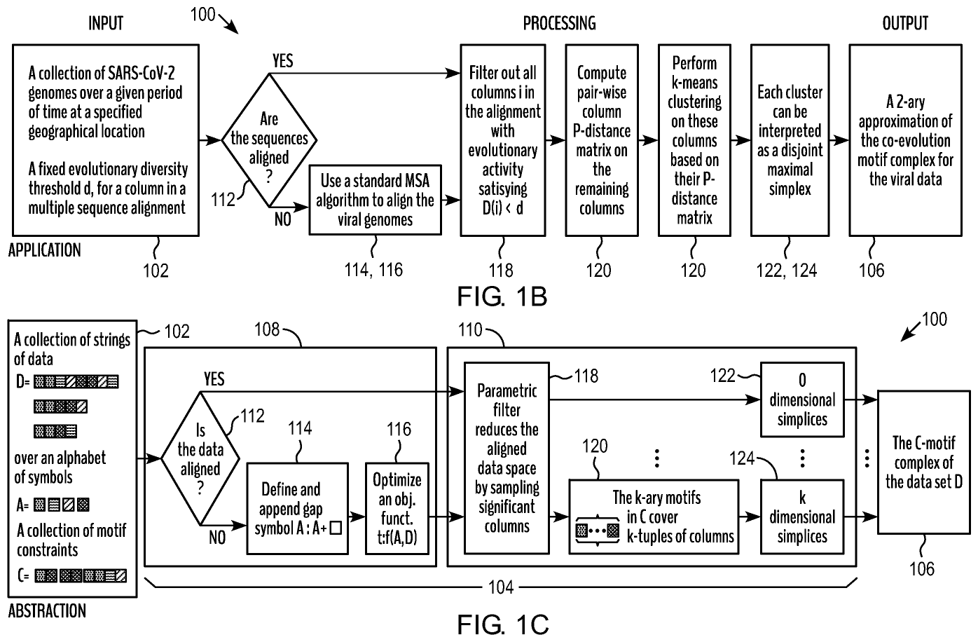

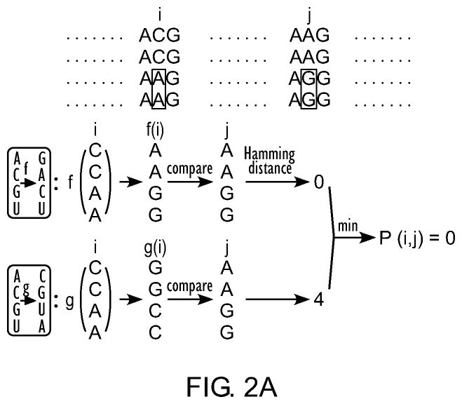

In exemplary embodiments, two components reflect the multiscale evolving nature of the bio-networks involved (see FIGS. 1A-1C):
1) Understanding the feedback loop between the macro-level social dynamics and the micro-level viral population composition during an epidemic.

Multiple layers of networks are needed to represent social, economic and viral interactions. This module employs transfer entropy concepts (TE) rooted in information theory. Algorithms and high performance computing simulations augment epidemic data to achieve robust measurements and quantifying perturbations at the macro scale by varying social policies.
2) Employing homological algebra to quantify phenotypic change and derive a notion of evolutionary paths connecting viral sequence-structure pairs via a biologically meaningful notion of “closeness”. The evolutionary capacity of a virus population as it evolves under various pressures induced by macro scale actions is defined.

### 1 TE Analysis:

Pursuant the first component, the viral population adapts to change in the behavior of the human population caused by the presence of the viral population itself. A macro-micro feedback loop within the multiscale bio-network of the COVID-19 pandemic thus becomes apparent.

During an epidemic, because macro-level dynamics vary with geographical location, distinct selective pressures are being exerted. Applicants have discovered that these pressures modulate the viral mutational landscape and lead to distinct geospatially separated mutational signatures, with the viral population composition responding very rapidly to certain macro-level dynamics. To assess this effect, TE measurements between macro and micro data for CA and NY respectively, quantified the amount of directed (time-asymmetric) transfer of information (in the sense of Renyi) between the two random processes. The viral populations of SARS-CoV-2 of CA and NY are determined to be causally linked (to a high degree of statistical significance) to their respective stale policies concerning social contact and workplace mobility.

Exemplary embodiments build on the following components: scalable distributed data structures for representing these networks as knowledge graphs; multi-programming scalable methods for simulating the co-evolution of viral dynamics; statistical methods to assess viral spread sensitivity and uncertainty quantification; and simulation-assisted epiviral workflows that support dynamic strain mutations.

### 2 Homological Accessibility:

Pursuant the second component, to properly understand the feedback mechanism across the micro-macro scales, one needs an adequate framework that characterizes structural nearness of the sequence-structure pairs within a given viral population. A problem arises due to fact that although evolution on the level of genomic sequences subjected to iterated point mutations is arguably continuous, no such continuity can be observed on the level of phenotypes.

Loops are the fundamental building blocks of viral RNA secondary structures, such as [13] M S Waterman. “Secondary structure of single-stranded nucleic acids”. In: Adv. Math. Suppl. Studies I (1978), pp. 167-212. To study the phenotypic accessibility between two such structures having the same number of nucleotides, a bi-structure {S.T) is employed. In [7] A C Bura, Q He, and C M Reidys. “Weighted Homology of Bi-Structures over Certain Discrete Valuation Rings, Mathematics 2021, 9(7), 744; https://doi.org/10.3390/math9070744, received: 23 Feb. 2021/Revised: 27 Mar. 2021/Accepted: 29 Mar. 2021/Published: 31 March 202, a homological analysis of bi-structures is used, where a loop is a set of nucleotides, and loop intersections are encoded via a simplicial complex called the loop nerve.

The nerve of a bi-structure has only the following nontrivial homology groups: Ho=Z, =©£=i Furthermore, the W\hat{ }-rank is generated by crossing components, i.e. specific combinatorial substructures that correspond to spheres in a wedge sum. This construction can be generalized to weighted complexes which leads to a novel boundary map: let ft I) Z be a discrete valuation ring with uniformizer K and denote by X the loop nerve of the bi-structure. Exemplary embodiments define v: X—>ft, such that for each a={c<i, . . . ,«*} 6 X. v(cT)=7r“^{lf}”, where ft>(cO:=|n,«;| is the number of nucleotides that lies in the mutual intersection of loops in o. The map d,′,: C,₁r(X)—>C,,_i.#(X), where (o)=£″₌₀(−1 allows a showing that a resulting homology of weighted complexes reduces to standard homology when setting all weights to one and augments the standard homology by introducing additional torsion modules that draw a refined picture of the intersection structure within the complex. For instance, in the augmented case. Ho is a torsion module whose invariant factors describe a certain minimal weight contraction procedure for a distinguished spanning sub-tree of the 1-skeleta of the weighted complex X. Intriguingly, the free rank of Wi reflects biological closeness:
1) all riboswitchcs in the Swispot database exhibit Wi-rank one.
2) the W? rank of a bi-structure obtained by two secondary structures of two sequences that are point mutants is at most one, stipulating a certain modularity assumption [10]. These facts point to a natural notion of continuity in phenotypic evolution captured by homology groups: a structure S is evolutionarily accessible (close) to another structure T only if r(H2(S.T)), the free rank of Hi of their respective bi-structure (S.T), is small.

A homology of weighted complexes is systematically developed and the interpretation of its torsion modules. Connections with parameterized complexity theories are applied for structure pairs that satisfy a single sequence from the perspective of theoretical computer science and hypergraphs. Furthermore, nearness concepts induced by a parameter of the augmented homology are identified, such as for example the H₂-rank, in which evolutionary transitions become Lipshitz continuous. A sensible notion of likely evolutionary trajectories is derived using H₂-rank as an ingredient for path-integral like notions. The sequence-structure pairs that can be attained in the course of evolution as well as whether or not certain sequences were bio-engineered, can be addressed by employing such paths.

The model of epidemic spread on multiscale social networks with evolution of different strains can be applied; see, for example, R Eletreby et al. “The effects of evolutionary adaptations on spreading processes in complex networks”. In: Proceedings of the National Academy of Sciences 117 (2020), p. 201918529 [8]. Epidemic thresholds and their fitness landscape can be exploited. Although in a document by S T Ali et al. “Serial interval of SARS-CoV-2 was shortened over time by nonpharmaceutical interventions”. In: Science 369.6507 (2020), pp, 1106-1109 [1]. The serial interval was investigated as a measure of the effectiveness of contact tracing and isolation.

Consider three strains of a compartmental SE1R disease. Strain 1 exhibits a high and equal rate of mutation into either strain 2 or strain 3. Strains 2 and 3 do not mutate and infection by any of the three strains confers immunity to all. In this example, a “lock-down” mitigation prevents any strain from spreading beyond a household. If the intervention lasts for k serial intervals, only households with more than i members can serve as reservoirs of disease. The viral parameters are such that a lock-down exerts selective pressure against strain 2, and as such the consequences depend on the distribution of household sizes. When the lock-down is released, strains 1 and 3 can escape from the reservoir households into the general population, and strain 1 will continue to mutate into 2 and 3, but now strains 1 and 3 have established themselves with much higher prevalence than strain 2. Because the prevalence ratio of strain 3 to strain 2 is much larger than in the base case with no mitigation, the overall fatality rate is also higher—how much higher depends again, on the distribution of household sizes.

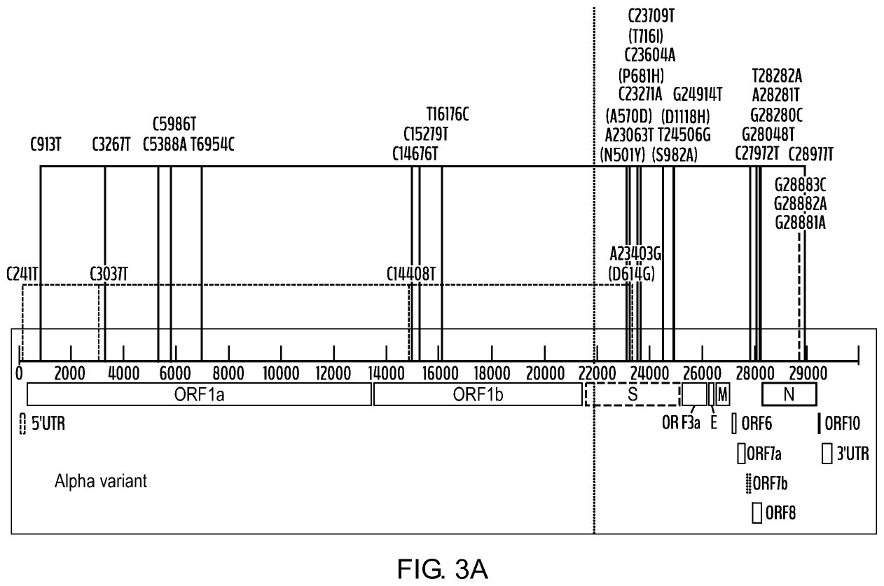

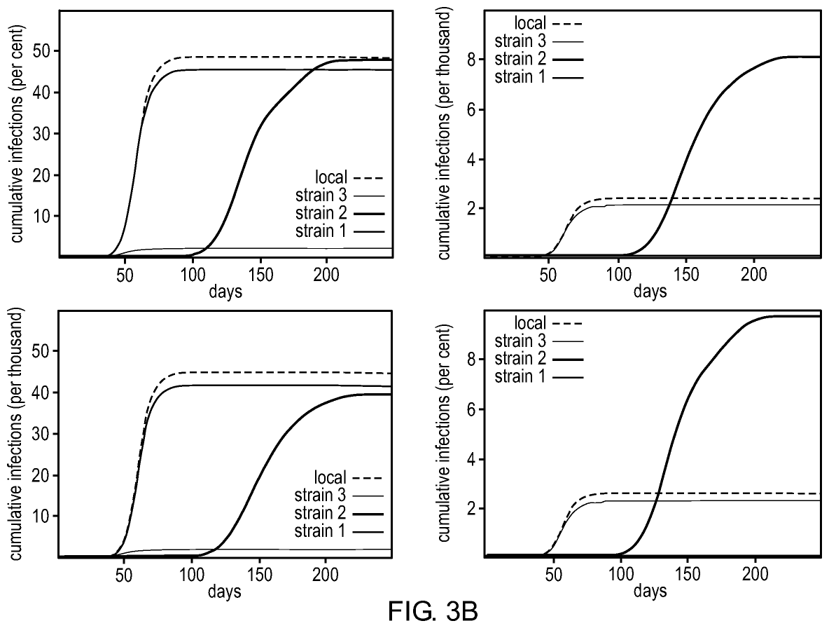

Moreover, the outcome of the competition among strains with or without lock down also depends on clustering in the graph. As demonstrated, a non-pharmaceutical intervention can exert selective pressure just as a pharmaceutical one does, but against different phenotypes, e.g. serial interval instead of epitope- and the consequences of that pressure depend on subtle relationships between the structures of the interaction network and the evolutionary phenotypic network. In an example, it is thus better not to select for strains with high evolutionary capacity (See FIGS. 3A and 3B). This experiment is intended not only to show that this effect is possible, but that a simulation system can generate the detailed consequences of the assumptions that will be necessary for really studying it.

The evolutionary capacity of the virus population involved in a simulation can be further determined. A virus population can be considered as a function ƒ:Q\hat{ }>N,f(0\.5,)=f, from the space of sequence structure pairs (2={(ft/.S,)} to their corresponding viral frequencies f, within the population. These frequencies will be incorporated in a notion of ‘topological search radii’ r(/,) w.r.t. a topology parameterized by an invariant of the homologies of weighted complexes of evolutionary paths (as per 2A} The union of open balls given by these radii will represent the evolutionary capacity of the examined viral population. Viral evolutionary capacity can be assessed from this perspective.

Referring to FIGS. 1A-1C, a data motif complex construction and application flow-charts, implemented as a computer-implemented software pipeline is illustrated, with an application for rapidly recognizing critical mutational blocks in viral SARS Covid2 geometric data specifically illustrated in FIG. 1B. In FIGS. 1B and 1C, collections of sites within coded sequences that are acted upon as blocks by selection pressure based on key (critical), informational patterns are determined.

FIGS. 1B and 1C illustrate a system and method 100 for efficient early detection, such as for indications and warnings, of co-evolutionary sites among aligned genomic sequences. The computer-implemented method can include receiving as an input 102 an aligned plurality of coded sequences, or aligning a plurality of coded sequences using a multiple sequence analysis (MSA) at an input of a computer processor 104. The processor 104 can have an optional specially programmed pre-processor 108 for aligning coded sequences and a specially programmed processor 110, which processors can of course be combined into a single hardware processor with multiple software modules, or implemented as multiple, dedicated and specially programmed processors. An output of the processor 104 can be supplied to a general user interface and/or display 106 for human observation and/or of implementing a user-specified biologic analysis (e.g., identifying indications and/or warnings of variants such as mutation variants of a virus or of the useful applications including, but not limited to assessing fitness of selected seed types for specified conditions such as draught resistance.

The pre-processor 108 can access whether input sequences are aligned in block/module 112, and if not, perform an alignment in any known fashion using for example, a define and append block/module 114 and an optimization block/module 116.

In the processor 110, blocks/module 118 is included for filtering columns of the aligned sequences wherein evolutionary activity satisfies an evolutionary diversity threshold (d); determining pair-wise column P- and/or J-distance matrices for remaining columns of the matrix subject to the evolutionary diversity threshold (d); and performing clusterings, such as k-means and HCS-clustering on the remaining columns using the P-distance matrix. Blocks/modules 122, 124 perform extracting of an m-ary approximation of a co-evolution data motif complex structure, the extracting or approximating including: constructing a vertex set of the data motif complex by identifying data sites with specified high informational variation; constructing specified high dimensional simplices which systematically represent key or critical informational patterns (e.g., patterns which satisfy a select condition or threshold as specified by the user and/or learned iteratively or via machine learning) within the data set; and determining and outputting collections of sites within the coded sequences that are acted upon as blocks by selection pressure based on the key or critical, informational patterns, for output to the GUI/display for biologic use and an analysis.

With reference to FIGS. 1B and 1C, a data point can often be abstracted as a string over a given alphabet, where each position corresponds to a feature of the data point with the entry in that site corresponding to a symbol that encodes the state of said feature. A collection of data points can thus be represented in a matrix form where each data point corresponds to a row. For example, a sample from a viral population for a given time and geographical location forms an alignment matrix A=(aᵢⱼ) composed of m sequences of length n. Here aᵢⱼϵΣ={e,A,C,G,T} represents the nucleotide in the jth position of the ith sequence, where e denotes a gap appearing in the sequence alignment.

Exemplary embodiments consider a k-tuple of A-columns i₁, i₂, . . . , iₖ where i₁<i₂< . . . <iₖ and query the existence of a k-ary relation between the respective nucleotides present at i₁, i₂, . . . , iₖ. Such a relation naturally appears if the specified k sites exhibit systematic dependencies among each other. In the case of genetic sequences, the relation can be considered to represent the existence of coevolution between these k positions. Note that exemplary embodiments consider a dependency of k sites that manifests via a variety of constellations of mutations.

For each collection of sites i₁, i₂, . . . , iₖ, such a relation among these sites corresponds to a set Mₖ[i₁, . . . , iₖ] consisting of k-tuples (aᵢ_{₁}, . . . , aᵢ_{ₖ}), which satisfies the following property: (aᵢ_{₁}, . . . , áᵢ_{ⱼ}, . . . , aᵢ_{ₖ})ϵM^{k-1}[i₁, . . . , îⱼ . . . , iₖ] for any jϵ{1, . . . , k}. Here âᵢ_{ⱼ} expresses the fact that aᵢ_{ⱼ} is omitted, i.e. any (k−1)-tuple induced by an Mₖ[i₁, . . . , iₖ] element, is a corresponding Mₖ₋₁[i₁, . . . îⱼ . . . , iₖ]-tuple. Exemplary embodiments refer to the collection X^{k}:=U_{[i}_{₁}_{, . . . , i}_{ₖ}_{]}Mₖ[i₁, . . . , iₖ], as k-motifs or motifs for short. The projectivity reflects the fact that, by construction, any subtnotif will be observed as an induced coevolutionary dependency.

Suppose the set of relations X:=UₖX^{k} is given. Then X:=UₖX^{k} gives rise to a weighted simplicial complex over the set of columns as follows: [i₀, . . . , iₖ] is a k-simplex of weight w if and only if |Mₖ[i₁, . . . , iₖ]|=w>0.

The particulars of all mutational constellations are projected onto their underlying sites.

It is natural to endow simplices with weights since the cardinality of Mₖ[i₁, . . . , iₖ] is an important factor in identifying the underlying coevolutionary dependencies in a given sample of aligned sequences.

The weighted complex formed by motifs and its algebraic homology theory allows one to express and quantify a variety of coevolution scenarios, in particular differentiating between the coevolution of three pairwise evolving columns [i,j], [j,k], [i,k] and that of three-wise coevolution [i,j,k]. Put differently, the framework allows distinguishing between empty and filled triangles.

An exemplary perspective is applied by which a distinguished family of motifs X a priori is presumed to exist, such that given multi-sequence alignment exhibits specific coevolution in accordance with the underlying motifs and processing the alignment row by row provides more and more information about the underlying coevolutionary relations. Depending on the composition of the sequence alignment and its size as well as potential errors introduced by constructing the multi-sequence alignment in the first place, and potential errors in the sequencing itself, the actual motif complex, i.e. the identification of all coevolutionary dependencies can only be approximated. In the following two such exemplary approximations are derived.

Multiple sequence alignment (MSA) is an algorithmic method to align multiple genetic sequences via inserting gap symbols, with the goal of identifying regions of genetic similarity. After alignment, the sequences are all of equal length and can thus be represented as a matrix with each sequence denoted by a row in the alignment matrix. Gaps symbols, which can be interpreted as indels, are inserted between the nucleotides so that identical or similar characters are aligned in successive columns. Each such column then represents a nucleotide position in the alignment, which (includes (e.g., consists) of nucleotides that are evolutionarily related.

Various efficient MSA algorithms have been developed and packaged such as: MAFFT, Clustal Omega, BLAST and HMMER, etc. These algorithms implement either precise optimizations or efficient heuristics in order to generate an alignment matrix that maximizes a similarity score for a given set of input sequence, and any one or more of these, or other functionally similar MSA, can be used individually or in combination, or in parallel.

Approximating the Data Motif Complex

To specify the complex for a given data set exemplary embodiments, a construct of its simplices starts from vertices (0-simplices) and proceeds upward in dimension to maximal simplices. In the case of viral evolution, these maximal simplices can be considered the key or critical simplices, since they represent collections of maximal, co-evolving positions, i.e. crucial functional units on the viral genome.

### Constructing 0-Simplices (Vertices)

The 0-simplices of the motif complex play a distinguished role in the evolutionary dynamics of the sequence sample. This manifests in two ways:
1. they are positions of competing variants within the multisequence alignment or
2. they are positions exhibiting significant variation for intrinsic, biochemical reasons.

Two exemplary ways to construct the 0-simplices are disclosed: one via nucleotide diversity and the other via Shannon entropy.

Nucleotide diversity: exemplary embodiments first compute the nucleotide diversity of a given column, i, in the alignment, i.e., and compute the column's average Hamming distance:

D(i)=(Σ_{1≤k<j≤m}Δₐ_{_{k,i}}_{,a}_{_{j,i}})/(₂^{m})

where Δ_{a,a′}=1-δ_{a,a′}, with δ_{a,a′} being the Kronecker symbol.

Shannon entropy: secondly exemplary embodiments employ the Shannon entropy, H(i), of a column i, given by

H(i)=−Σ_{xϵΣ}pᵢ(x)log₂pᵢ(x)

where the units of H are bits, and pᵢ(x) is the probability of the nucleotide x appearing in column i. This entropy has been widely utilized in order to quantify the nucleotide variation in a fixed column of a given alignment.

After computing D(i) (H(i)) for all columns i, the columns with D(i) or (H(i))>h₀ will be selected as 0-simplices.

### Constructing 1-Simplices (Edges)

Approximating the motif complex's 1-simplices, for output of the GUI/display 106, for biologic analysis, can be performed via either of two ways to construct them: one via P-distance, and/or the other via J-distance.

P-distance: consider all permutations τ:Σ→Σ and make the Ansatz

$$P ( i , j ) = \frac{1}{( \begin{matrix}m \\ 2\end{matrix} )} {min}_{τ} {\sum }_{k = 1}^{n} {Δ}_{τ ( {a}_{k , i} ) , {a}_{k , j}} .$$

In the context of a noisy data-set, P(i,j) can be viewed as a reverse-engineering of the permutations that induce the underlying dependencies between the two columns. Relations like identity or complementarity can readily be expressed via such mappings. P(i,j) satisfies by construction P(i,j)=P(j,i) and the triangle inequality P(i,h)≤P(i,j)+P(j,h). That is, P(i,j) is a pseudo-metric. In the case of the data being a genetic sequence alignment, there are only 5! permutations that need to considered, corresponding to four nucleotide types and a gap symbol, as such P(i,j) can be computed easily.

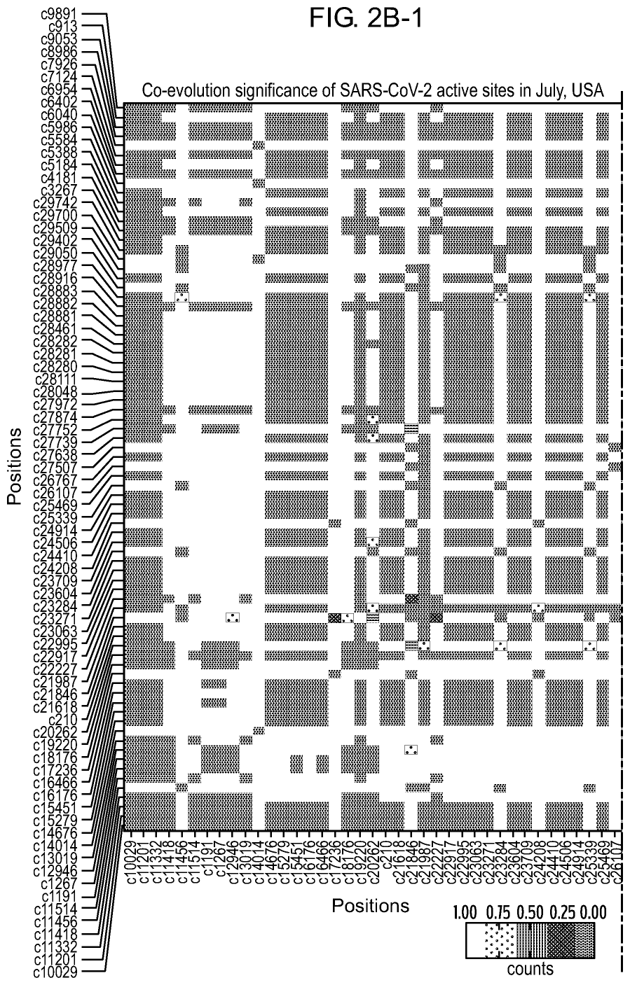

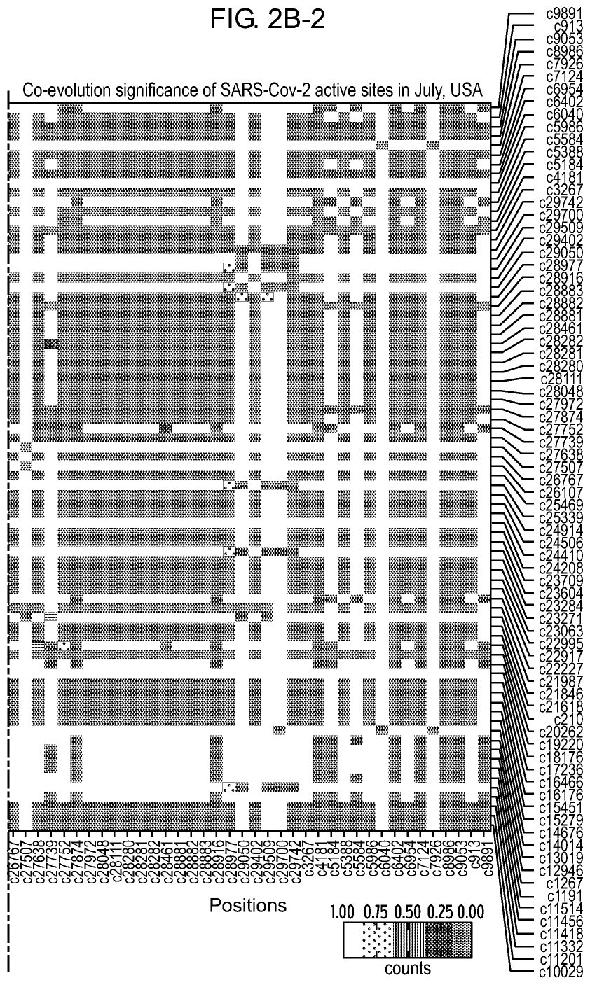

Note that P(i,j) is completely determined by the joint distribution p_{i,j}(x, y) of pairs of nucleotides, namely P(i,j)=1−max_{τ}p_{i,j}(x,τ(x)), as illustrated in FIG. 2A and below.

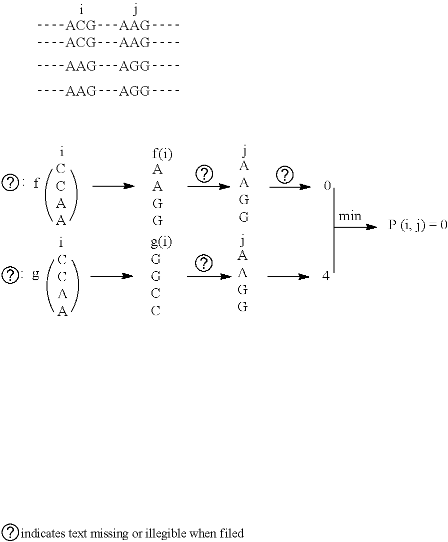

J-distance: an alternative approach to the P-distance is achieved via joint entropy and mutual information as follows: the joint entropy H(i;j) of two sites i and j is defined as

$$H ( i , j ) = - {\sum }_{x} {\sum }_{y} {p}_{i , j} ( x , y ) {log}_{2} {p}_{i , j} ( x , y ) ,$$

where p_{i,j} denote the joint distribution of columns i and j. I.e., p_{i,j}(x, y) specifies the probability of the pair of nucleotides (x,y) occurring in the column pair.

Clearly, the marginal probability distributions for columns i and j are given by pᵢ(x)=Σ_{y}p_{i,j}(x,y) and pⱼ(y)=Σₓp_{i,j}(x,y), respectively.

The mutual information I(i,j) between sites i and j is the relative entropy between the joint distribution p_{i,j}(x, y) and the product distribution pᵢ(x)pⱼ(y):

$$\begin{matrix}I ( i ; j ) = D ( {p}_{i , j} ( x , y ) ‖ {p}_{i} ( x ) {p}_{j} ( y ) ) \\ = {\sum }_{x} {\sum }_{y} {p}_{i , j} ( x , y ) {log}_{2} \frac{{p}_{i , j} ( x , y )}{{p}_{i} ( x ) {p}_{j} ( y )}\end{matrix} ,$$

where D(p∥q) denotes the relative entropy or Kullback-Leibler divergence from the distribution p to the distribution q. The mutual information I(i;j) quantifies the amount of information shared by the columns i and j.

Then the J-distance between two columns i and j represents the information-theoretic counterpart of the Jaccard distance and is given by:

$$J ( i , j ) = 1 - \frac{I ( i ; j )}{h ( i ; j )} .$$

Both the P- and J-distances are closely tied to the permutations generating the motifs. While this holds obviously for P(i,j), it follows for J(i,j) from the fact that if J(i,j)=0, then there exists a distinguished bijection τ on the alphabet such that, for each x, p_{i,j}(x,τ(x))=pᵢ(x)=pⱼ(τ(x)) and p_{i,j}(x,y)=0, otherwise.

After computing the P-distances (J-distances) for all column pairs, those with P-distance (J-distance) smaller than a given threshold E will be selected as 1-simplices.

### Statistical Significance of a P-Distance (J-Distance) Measurement:

A null hypothesis can be tested, which models the scenario that column i and j evolved independently. Observing a small P(i,j) or a J(i,j) value respectively, for a column pair (i,j), gives rise to a 1-simplex in the approximation. These values, however, have to be put into the context of their respective nucleotide frequencies. As such the null hypothesis will be:
“Columns i and j are drawn independently, each from a uniform distribution that consists of columns whose nucleotide frequencies are identical to those of i and j, respectively.”

For this exemplary null hypothesis p-values are computed that measure the likelihood of observing a P or a J measurement, which by chance is smaller than or equal to P(i,j) and J(i,j) respectively.

An approximation of the null distribution is constructed via the uniform sampling of a pair of (pᵢ(x))ₓ- and(pⱼ(x))ₓ-columns.

Firstly, the number of columns, c, exhibiting specific a (pᵢ(x))ₓ is

N=n!/Πnₙ!,

where nₓ denotes the number of nucleotides of type x in c. Uniformly sampling such (pᵢ(x))ₓ-columns is equivalent to uniformly sampling πϵSₘ and permuting the entries of a fixed (pᵢ(x))ₓ-column, c₀.

The probability of realizing any such (pᵢ(x))ₓ-column, via a random permutation is N⁻¹, which equals the probability of a(pᵢ(x))ₓ-column in the sample space. By construction

P(π₁(i),π₂(j))=P(π₁π₂⁻¹(i),j)=P(i,π₂π₁⁻¹(j)).

Similar result holds for J-distance, namely:

J(π₁(i),π₂(j))=J(π₁π₂⁻¹(i),j)=J(i,π₂π₁⁻¹(j)).

Accordingly, the uniform sampling of a pair of (pᵢ(x))ₓ- and (pⱼ(x))ₓ-columns can be facilitated by uniformly sampling permutations πϵSₘ of either i or j and considering the pair (i,π(j)) as a sample in the null distribution. Fixing by the aforementioned process, a suitable sample set S of pairs from the null distribution, provides for a computation of the ratios:

$$\begin{matrix}{p}_{i , j} ( P ) = \frac{\vert \{ ( i , π ( j ) ) \vert πϵ {S}_{m} , P ( i , π ( j ) ) \leq P ( i , j ) \} \vert}{\vert S \vert} \\ {p}_{i , j} ( J ) = \frac{\vert \{ ( i , π ( j ) ) \vert πϵ {S}_{m} , J ( i , π ( j ) ) \leq J ( i , j ) \} \vert}{\vert S \vert}\end{matrix} .$$

These ratios approximate the exemplary p-values of interest. Small values of these ratios refute the null hypothesis, providing a degree of sensitivity of measurements on the pair (i,j).

### Higher Dimensional Simplices

Based on P(i,j) or J(i,j) for any pair of vertex columns can approximate higher dimensional simplices can be approximated for output by performing clustering (block module 122, 124), via for example, 1-means clustering.

K-means clustering is an unsupervised machine learning algorithm of vector quantization, aiming to partition n observations into k clusters in which each observation belongs to the cluster with the nearest mean to the cluster center. The problem is in general NP-hard, but efficient heuristic algorithms are available and can achieve a local optimum. As the number of clusters k is part of the input, the first step is to determine a suitable k. Here, the k-means clusters are computed using different values for the number of clusters k, k<30. Then a wss (within sum of squares) distribution is computed according to k. The location of a bend in the distribution is generally considered as an indicator of the appropriate number of clusters. By construction, the exemplary method is conducive to an optimal cluster number that does not vary too much with increases in the k range. This is since clusters (and their sizes) are self similar only for truly uniform data points—in which case the optimum cluster number would drift depending on the interval chosen. The analysis is processed by the factoextra package in R.

Each cluster in the clustering result of the k-mean algorithm can then be interpreted in block/module 122,124 as a maximal simplex in the approximation of the Data Motif Complex.

An alternative approach to constructing k-simplices for k>1 is to extract highly connected subgraphs of a similarity graph induced by P(i,j) (J(i,j)) and a threshold value, via the HCS-algorithm.

### Implementation

This computer-implemented approximation system/method can be implemented in for example, C as command line tools. The C package can be robustly compiled and installed on multiple computer platforms. For example, a SARS-CoV-2 genetic analysis performed by this methodology was implemented on specially programmed Mac workbooks and the Rivanna HPC cluster at UVA.

### Application to Rapid Recognition of Critical Mutational Blocks

As a general method, exemplary embodiments can be used to detect the dependency structure within a given data set. For example, the method can facilitate biologic analysis such as genomic sequence analysis. Potential other applications may include: detection of engineered genetic sequences, identification of gene regulatory networks to guide biological experiments, detection of emergence of viral variants etc. An application for the rapid recognition of critical mutational blocks in SARS-CoV-2 viral populations, which facilitates early threat detection of viral variants of concern, is already discussed.

### Data Preparation

For the experimental implementation described in FIG. 1B, high-quality SARS-CoV-2 whole genome data was collected from GISAID ([15] Elbe and Buckland-Merrett, 2017, Data, disease and diplomacy: Gisaid's innovative contribution to global health. Global Challenges 1 (1), 33-46; Shu and McCauley, 2017, Gisaid: Global initiative on sharing all influenza data—from vision to reality. Eurosurveillance 22 (13). WHO, 2021. WHO announces simple, easy-to-say labels for SARS-CoV-2 variants of interest and concern. www.who.int) on a regular basis (the latest one is collected at 4th, September). Each sequence is individually aligned to the reference sequence collected from Wuhan, 2019 (GISAID ID: EPI ISL 402124), using the multiple sequence alignment algorithm MAFFT: a method for rapid multiple sequence alignment based on fast fourier transform. Nucleic acids research 30 (14), 3059-3066. (Katoh et al., 2002). Specifically, both duplicate and low-quality sequences (>5% NNNNs) have been removed, using only complete sequences (length>29,000 bp). The resulting alignment of SARS-CoV-2 sequences is created using MAFFT (access via https://doi.org/10.1093/molbev/mst010) in 3 separate steps.
1. Each sequence is individually aligned to the reference Wuhan sequence hCoV-19/Wuhan/WIV04/2019 (GISAID ID: EPI ISL 402124). Sequences that created dubious insertions of >12 nucleotides in the reference sequence and occurred only once in the database are discarded. The alignments are created with the command:
   mafft—thread-1 input. fasta>output. fasta
2. All sequences that result in insertions in the reference from step 1 are then aligned with a opening gap penalty of 10 to prevent long stretches of dubious insertions in the alignment due to the presence of long stretches of NNNNs. The following command is used:
   $ mafft—retree 3—maxiterate 10—thread-1—nomemsave—op 10 seqsCausingInsertionsInRef.fasta>seqs_aligned.fasta
3. The rest of the sequences that did not result in insertions are aligned to the resulting alignment in step 2 with this command:
   $ mafft—thread 1—quiet—keeplength—add
   sequencesNotCausingInsertionsInRef.fasta seqs_aligned.fasta>msa_0830.fasta

The sequences in the alignment are then partitioned into bins according to the months and the locations they were collected on. This partition process is based on the meta information associated with each sequence record. In our following analysis, exemplary embodiments consider the sequences in each month at a specific location as a viral population.

The defining mutations of VOCs: Alpha, Beta, Gamma, Delta, Epsilon, Kappa were collected together with EU1 (also known as B.1.177, spread widely across Europe in the summer of 2020) and AY. 3&4 (two sub variants of the Delta variant, spread fast in US and UK) from (WHO, 2021; Brown et al., 2021 (Brown, C., Vostok, J., Johnson, H., et al., 2021. Outbreak of SARS-CoV-2 infections, including covid-19 vaccine breakthrough infections, associated with large public gatherings. Morbidity and Mortality Weekly Report 70 (31), 1059-1062, DOI: http://dx.doi.org/10.15585/mmwr.mm7031e2); Hodcroft et al., 2021 (Hodcroft, E. B., Zuber, M., Nadeau, S., Vaughan, T. G., Crawford, K. H. D., Althaus, C. L., Reichmuth, M. L., Bowen, J. E., Walls, A. C., Corti, D., Bloom, J. D., Veesler, D., Mateo, D., Hernando, A., Comas, I., Gonzalez Candelas, F., Stadler, T., Neher, R. A., 2021. Spread of a SARS-CoV-2 variant through Europe in the summer of 2020. Nature 595 (7869), 707-712).

### Data Stratification

Filtering SARS-Cov-2 whole genome sequences from the GISAID multiple sequence alignment data yields SARS-Cov-2 whole genome sequence with specified temporal and geographic information.
Input: GISAID multiple sequence alignment data
Output: SARS-Cov-2 whole genome sequence for specified temporal and geographic information
Function:
filter_covid [-OPTIONS]
[-h] Help
[-i inputfile] Specify the input file. (FASTA format)
[-o output file] Specify the output file.
[-m month] Specify the filter month. (Oct2019=1, Jan2020=4, Jan2021=16)
[-w week] Specify the filter week. (week1=1-7, week2=8-15, week3=16-23, week4=23—)
[-r region] Specify the filter region. (Europe, SouthAmerica, Aisa, NorthAmerica, Africa, Oceania)
[-c country] Specify the filter region.

### Example

./covid filter, -i msa_0814.fasta-o USATimeBin22.fasta-m 22, -w 3, -c USA

### D(i) Calculation and Filtration

Given the alignment of sequences collected above with respect to a selected month and a geographical location, D(i) was computed for each site and output a list of columns with D(i)>d, where d is a fixed evolutionary diversity threshold. In our analysis d=0.1. This will produce the set of vertices in the approximation of the Motif Complex for the SARS-CoV-2 genomic data.
Input: SARS-Cov-2 whole genome sequence for specified temporal and geographic information
Output: A list of active positions with D(i) greater than a given threshold.
Function:
read covid diversity [-OPTIONS]
[-h] Help
[-i inputfile] Specify the input file. (FASTA format)
[-o output file] Specify the output file.
[-t threshold] Specify the D(i) threshold.

### Example

./read covid diversity-i USATimeBin22.fasta-o
USATimeBin22_column_diversity_010.out-t 0.1

### P-Distance Calculation

The pairwise P-distance is computed from the list of active positions. The distance facilitates the construction of edges in the approximation of the Motif Complex for the SARS-CoV-2 genomic data.
Input: A list of active positions on the SARS-CoV-2 genome.
Output: A matrix with pairwise P-distance for the list of active positions, whose (i,j) entry is the p-distance between column i and j. An optional parameter can switch between p-distance and Jaccard distance as needed.
Function:
pdis [-OPTIONS]
[-h] Help
[-i inputfile] Specify the whole genome sequence input file. (FASTA format)
[-c active_position] Specify the list of active positions on the SARS-CoV-2 genome
[-o output file] Specify the output file.
[-p pos_map] Specify the position map information file for the reference sequence.

### Example

./pdis USATimeBin22.fasta-c USATimeBin22_column_diversity_010. out-o USATimeBin22_distance.out

### Statistical Testing

The statistical significance of P-distance for all pairs within a list of active positions is computed.
Input: A list of active positions on the SARS-CoV-2 genome.
Output: A matrix with pairwise p-value for the list of active positions.
Function:
stat [-OPTIONS]
[-h] Help
[-i inputfile] Specify the whole genome sequence input file. (FASTA format)
[-c active_position] Specify the list of active positions on the SARS-CoV-2 genome
[-o output file] Specify the output file.

### Example

./stat USATimeBin22.fasta-c USATimeBin22_column_diversity_010.out-o USATimeBin22 stat.out

### Clustering

The k-means clustering is for example, computed from a matrix with pairwise P-distance by utilizing the R package “factoextra”. This will provide us with the maximal simplices in the approximation of the Motif Complex for the SARS-CoV-2 genomic data
Input: A matrix with pairwise P-distance.
Output: Clustering information

### Example

library(“factoextra”)
Import data to R:
>USA_coevol_Bin22<-read.csv(“USATimeBin22_distance.out”, sep=“ ”)
Estimate the optimal number of clusters k:
>fviz_nbclust(USA_coevol_Bin22, kmeans, k.max=10, method=“gap stat”)
Compute the k-means clusters:
>set.seed(123)
>USA_coevol_Bin22.km<—kmeans(USA_coevol_Bin22, 7, nstart=25)
Output: USA_coevol_Bin22.km

### Results

FIGS. 2B-1 and 2B-2 show an exemplary heat map of p-values for P(i,j) of active sites, July 2021, USA.

### Retrospective Study of the Alpha Variant in England

The Alpha variant was first detected in England in November 2020, with reference to FIG. 3A. This variant contains 27 mutations that are commonly observed within the variant genome (>90%). Ccollect SARS-CoV-2 whole genome data for the month of November 2020, in England, from GISAID. In the first experiment, the method to cluster the aforementioned mutations via k-means and P-distance. The clustering results into three groups, see FIG. 3A which provide a more refined analysis of these mutations as follows: among these three groups is one containing the C241T, C3037T, C14408T, and A23403G mutations. These four mutations were previously observed in February 2020 and began dominating the population by April 2020. As such, they are of low evolutionary activity, having a low D(i), and their corresponding cluster can thus be discounted.

Similarly, the second of these groups contains G28881A, G28882A, G28883T, all three of which are known to be mutations that have emerged before the Alpha variant was detected. As such, on the basis of our method, this cluster can also be discounted. The remaining 20 mutations form the third and final group as shown in FIG. 3A, and represent the characteristic signature of the Alpha variant.

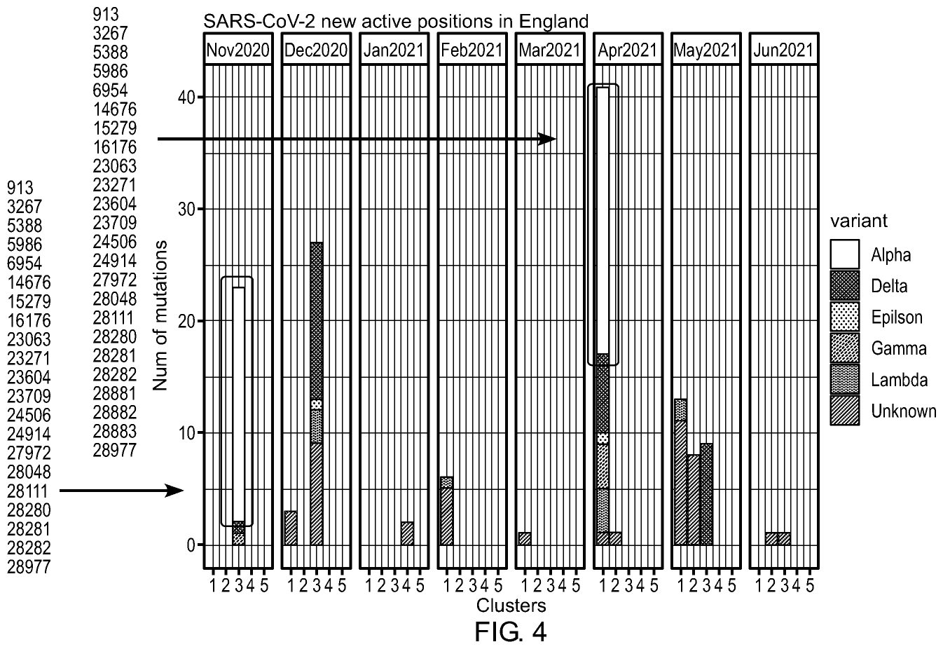

In a second experiment on the England data is shown in FIG. 3A regarding a study of the emergence of active mutational positions (D(i)>0.1), month by month, from November 2020 to June 2021. FIG. 4 presents the number of new active mutations emerging when compared to the previous month. The total number of clusters is kept for each month constant (5) introducing empty clusters if in a particular month the number of clusters generated is smaller than 5. Each colored portion of a vertical bar represents the ratio of actively emerging mutations corresponding to various variants (that are similarly color coded) within each given cluster. If a mutation is contained in multiple variants then it is counted with multiplicity in the figure.

Timely identification of the Alpha variant: although in November 2020 the prevalence within the viral population of the Alpha variant is relatively low in England (<5%) and the variant was not yet declared to be of concern, and already observed the emergence of a co-evolving cluster (the third cluster from the previous experiment) of positions that match the Alpha variant. This suggests that our method is highly sensitive and capable of providing early warning for the emergence of VoC. Alpha variant steady state: FIGS. 3A and 3B show that, as the variant establishes itself, the previously mentioned positions are no longer evolutionarily explored (December 2020 through March 2021). Alpha variant reactivation: finally, FIG. 4 shows that the same positions become active again, because of evolutionary pressures introduced by its competition with the novel Delta variant which is rapidly establishing dominance within the viral population.

### Rapid Identification of SARS-CoV-2 Motifs in the USA

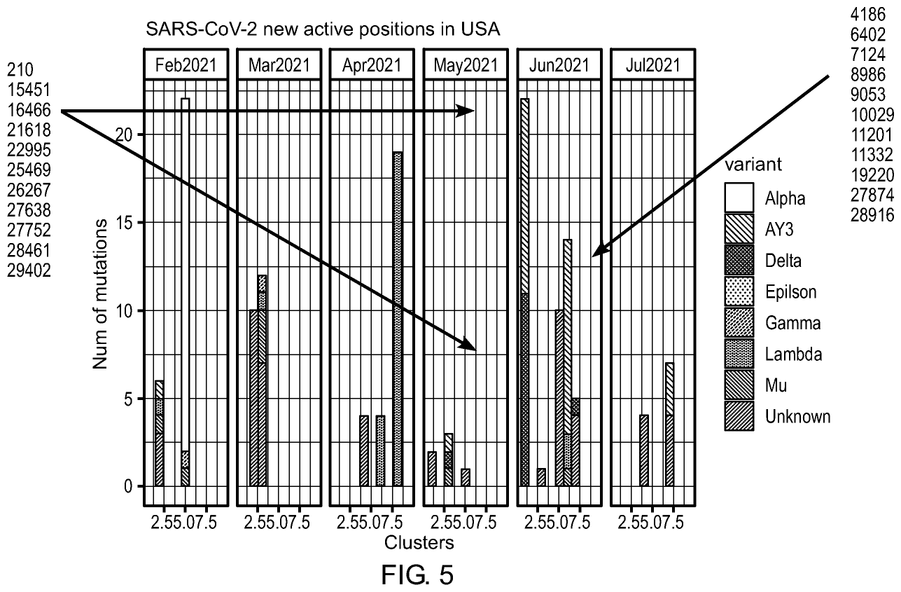

A similar type of analysis was performed as in the previous section but for USA data collected between February 2021 to July 2021, with reference to FIG. 3B. Coevolution analysis of AY.3 lineage: although in June 2021 the prevalence within the viral population of the AY.3 lineage is relatively low in England (<5%) and the variant was not yet declared indexed, one can observe the emergence of two co-evolving cluster corresponding to AY.3, see FIG. 5. The first of these, also contains the Delta variant relevant positions. This is since AY.3 is a sub-lineage of Delta. Delta variant development: the second cluster observed in FIG. 5 does not overlap with Delta variant positions being an independent mutational block. This supports the notion that the AY.3 variant is the outcome of the Delta variant being subjected to evolutionary pressures and exploring new adaptations.

### Rapid Identification of SARS-CoV Motifs in South America

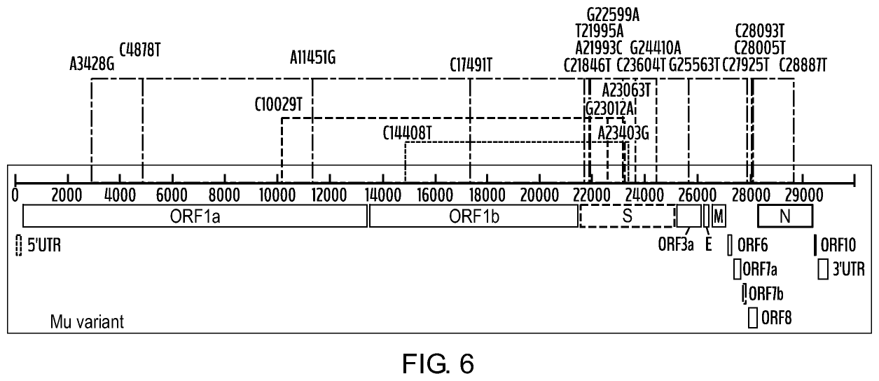

The Mu variant referenced in FIG. 6 is a newly declared VoI (August 2021) being first detected in South America and Europe. Most of the relevant mutations that comprise Mu are also found in previously declared VoCs, such as Alpha and Gamma. Exemplary embodiments perform a similar type of clustering for data collected for the month of July 2021 in South America, see FIG. 6. The mutations of the Mu variant co-evolve to a high degree, most of them presenting in a single cluster of size 15 while the remaining 5 two smaller clusters of sizes 3 and 2 respectively, with the smallest of them corresponding to the D614G Glade.

Although the mutational positions corresponding to the large cluster of Mu are not novel, as all of them appear in other VoCs, the co-evolution pattern produced by our method implies that the Mu variant is the outcome of synchronization between mutations within the viral population corresponding to previously disparate variants.

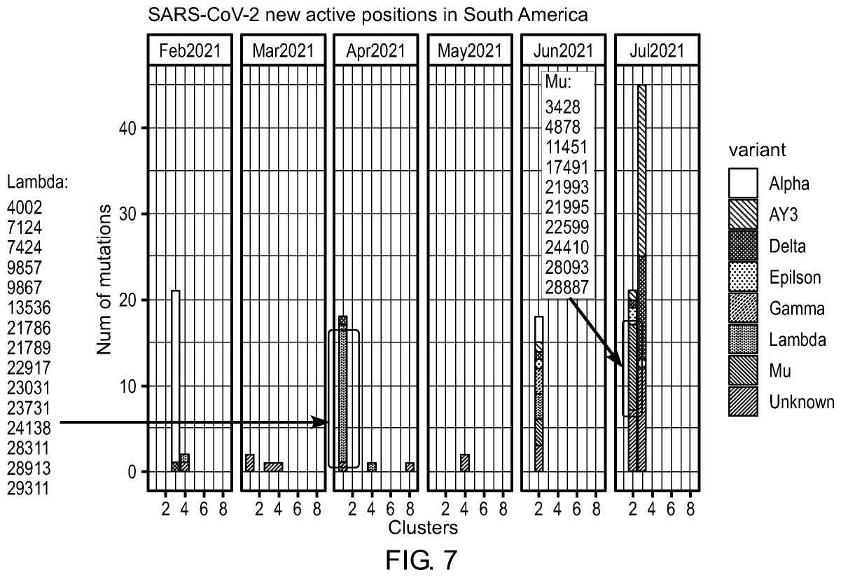

FIG. 7 displays, as before, emerging active mutational positions, month by month in, data collected between February 2021 and July 2021 in South America. Timely identification of the Lambda variant: although in April 2021 the prevalence within the viral population of the Lambda variant is relatively low in South America(<5%) and the variant was not yet declared to be of concern, and already observed the emergence of a co-evolving cluster of positions that match the Lambda variant, see FIG. 6. Mu precursor: FIG. 6 also shows that in June 2021 a cluster of positions corresponding to the following variants activates: Alpha, Delta, Epsilon, Gamma, Lambda. This suggests the viral population is performing preparatory explorations for the emergence of the Mu variant, while in July 2021 exemplary embodiments observe a cluster corresponding to the emergence of the Mu variant itself. This cluster is disjoint from the Delta corresponding cluster, suggesting that the Mu variant is a direct competitor to Delta.

The foregoing has been described in the context of exemplary embodiments directed to pandemic indications and warnings. The exemplary applications include, for example, determining indications and warnings of possible pandemic warning indications for assisting with predicting and planning a response to global phenomena such as biocomplex pandemics. However, exemplary embodiments can be applies to any of a number of applications readily apparent to those skilled in the art. For example, embodiments as described herein can be used for assessing genetic make-to assess where on a genome a putative fitness exists when evaluating fitness of selected seed types for specified conditions such as draught resistance.

A non-transitory computer readable medium, the computer readable medium storing program code for performing data processing, the program code causing a processor to perform operations as disclosed. A person having ordinary skill in the art would appreciate that embodiments of the disclosed subject matter can be practiced with various computer system configurations, including multi-core multiprocessor systems, minicomputers, mainframe computers, computers linked or clustered with distributed functions, as well as pervasive or miniature computers that can be embedded into virtually any device. For instance, one or more of the disclosed modules can be a hardware processor device with an associated memory.

A hardware processor device as discussed herein can be a single hardware processor, a plurality of hardware processors, or combinations thereof. Hardware processor devices can have one or more processor “cores.” The term “non-transitory computer readable medium” as discussed herein is used to generally refer to tangible media such as a memory device.

Various embodiments of the present disclosure are described in terms of an exemplary computing device. After reading this description, it will become apparent to a person skilled in the relevant art how to implement the present disclosure using other computer systems and/or computer architectures. Although operations can be described as a sequential process, some of the operations can in fact be performed in parallel, concurrently, and/or in a distributed environment, and with program code stored locally or remotely for access by single or multi-processor machines. In addition, in some embodiments the order of operations can be rearranged without departing from the spirit of the disclosed subject matter.

A hardware processor, as used herein, can be a special purpose or a general purpose processor device. The hardware processor device can be connected to a communications infrastructure, such as a bus, message queue, network, multi-core message-passing scheme, etc. An exemplary computing device, as used herein, can also include a memory (e.g., random access memory, read-only memory, etc.), and can also include one or more additional memories. The memory and the one or more additional memories can be read from and/or written to in a well-known manner. In an embodiment, the memory and the one or more additional memories can be non-transitory computer readable recording media.

Data stored in the exemplary computing device (e.g., in the memory) can be stored on any type of suitable computer readable media, such as optical storage (e.g., a compact disc, digital versatile disc, Blu-ray disc, etc.), magnetic tape storage (e.g., a hard disk drive), or solid-state drive. An operating system can be stored in the memory.

In an exemplary embodiment, the data can be configured in any type of suitable database configuration, such as a relational database, a structured query language (SQL) database, a distributed database, an object database, etc. Suitable configurations and storage types will be apparent to persons having skill in the relevant art.

The exemplary computing device can also include a communications interface. The communications interface can be configured to allow software and data to be transferred between the computing device and external devices. Exemplary communications interfaces can include a modem, a network interface (e.g., an Ethernet card), a communications port, a PCMCIA slot and card, etc. Software and data transferred via the communications interface can be in the form of signals, which can be electronic, electromagnetic, optical, or other signals as will be apparent to persons having skill in the relevant art. The signals can travel via a communications path, which can be configured to carry the signals and can be implemented using wire, cable, fiber optics, a phone line, a cellular phone link, a radio frequency link, etc.

It will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein.

1. A method for efficient early detection co-evolutionary sites among aligned genomic sequences, the method comprising:
filtering columns of aligned coded sequences wherein evolutionary activity satisfies an evolutionary diversity threshold (d);
determining a pair-wise column P-distance matrix for remaining columns of the matrix subject to the evolutionary diversity threshold (d);
performing on the remaining columns using the P-distance matrix; and
extracting an m-ary approximation of a co-evolution data motif complex structure, the extracting or approximating including:
constructing a vertex set of the data motif complex by identifying data sites with specified high informational variation; constructing specified high dimensional simplices which systematically represent key or critical, informational patterns within the data set; and
determining and outputting collections of sites within the coded sequences that are acted upon as blocks by selection pressure based on the key or critical, informational patterns.

2. The method for early detection as claimed in claim 1, comprising:
performing the clustering as k-means and/or HCS-clustering; and
aligning a plurality of coded sequences using a measurement systems analysis (MSA).

3. The method for early detection as claimed in claim 1, comprising:
determining pair-wise column P- and J-distance matrices for remaining columns of the matrix subject to the evolutionary diversity threshold (d).

4. The method for early detection as claimed in claim 1, comprising:
detecting variants for indication and warnings during biologic analysis.

5. The method for early detection as claimed in claim 1, comprising:
assessing a collection represented by a population of RNA nucleotide sequences associated with positive samples of an infectious population.

6. The method for early detection as claimed in claim 1, comprising:
assessing a collection represented by a population of DNA sequences associated with positive samples of an infectious population.

7. The method for early detection as claimed in claim 1, comprising:
implementing the method as a software pipeline.

8. The method for early detection as claimed in claim 1, comprising:
recognizing maximal critical blocks within variants in viral SARS-CoV-2 genomic data.

9. The method for early detection as claimed in claim 1, wherein each cluster is interpreted as a disjoint maximal simplex.

10. A system for efficient early detection of co-evolutionary sites among aligned genomic sequences, the system comprising a computer programmed to perform the steps of:
filtering columns of aligned coded sequences wherein evolutionary activity satisfies an evolutionary diversity threshold (d);
determining a pair-wise column P-distance matrix for remaining columns of the matrix not subject to the evolutionary diversity threshold (d);
performing clustering on the remaining columns using the P-distance matrix; and
extracting an m-ary approximation of a co-evolution data motif complex structure, the extracting or approximating including:
constructing a vertex set of the data motif complex by identifying data sites with specified high informational variation;
contracting specified high dimensional simplices which systematically represent informational patterns within the data set; and
determining collections of sites within the coded sequences that are acted upon as blocks by selection pressure based on the key or critical, informational patterns; and
a display for outputting a detected variant.

11. The system for early detection as claimed in claim 10, wherein the computer is programmed to perform the step of:
performing the clustering as k-means and/or HCS-clustering; and
aligning a plurality of coded sequences using a measurement systems analysis (MSA).

12. The system for early detection as claimed in claim 10, wherein the computer is programmed to perform a step of:
detecting variants for indication and warnings facilitating effective biologic analysis.

13. The system for early detection as claimed in claim 10, wherein the computer is programmed to perform a step of:
assessing a collection represented by a population of RNA nucleotide sequences associated with positive samples of an infectious population.

14. The system for early detection as claimed in claim 10, wherein the computer is programmed to perform a step of:
assessing a collection represented by a population of DNA sequences associated with positive samples of an infectious population.

15. The system for early detection as claimed in claim 10, wherein the computer is programmed to perform a step of:
implementing the method as a software pipeline.

16. The system for early detection as claimed in claim 10, wherein the computer is programmed to perform a step of:
detecting critical mutational blocks in viral SARS-CoV-2 genomic data.

17. The method for early detection as claimed in claim 1, wherein each cluster is interpreted for indications and warnings of variants associated with pandemic mutations.

18. The method for early detection as claimed in claim 1, wherein each cluster is interpreted for indications and warnings of variants associated with specified seed fitness.
